(19) United States
(12) Patent Application Publication
(10) Pub. No.: US 2004/0267562 A1
(43) Pub. Date: Dec. 30, 2004

(54) Method for taking a sample from a system

(76) Inventors: Thomas Fuhrer, Hagenberg (AT); Robert Schossleitner, Ampflwang (AT)

Correspondence Address: WILLIAM COLLARD, COLLARD & ROE, P.C., 1077 NORTHERN BOULEVARD, ROSLYN, NY, 11576, US

(21) Appl. No.: 10/488,669

(22) PCT Filed: Sep. 5, 2002
(86) PCT No.: PCT/AT02/00257

(30) Foreign Application Priority Data
Sep. 5, 2001 (AT) A 1402/2001

(51) Int. Cl. G06F 17/60; G06F 19/00; G01N 33/48; G01N 33/50; A61B 5/00; G06F 17/00

(52) U.S. Cl. 705/2; 235/375; 702/19; 600/301

(57) ABSTRACT

The invention relates to a method for taking a sample from a system. A user (1) enters an analysis request to a computer system (4), whereupon a connection is established between the computer system and a databank. As a result of the analysis request an information catalogue is generated from the databank, from which the at least one sample holder (13) to be used is suggested or provisionally selected for the user (1) by means of a input and/or output device (5). The invention further relates to a method for production of a request data set for a sample for analysis. An information catalogue, corresponding to an entered analysis request is proposed to the user (1), the data of which from the information catalogue for the entered analysis request may be optionally added to or edited by the user (1). From said data, test conditions for the sample to be analysed are generated and processed with the holder data, a system identification number and a request number to give a uniform machine-processable request data set which is transmitted to the laboratory (2) engaged for the above.

The invention relates to a method for taking a sample from a system and also to a method of generating order records for at least one sample to be analysed, according to the features in the introductory clauses of claims 1 and 2.

As is generally known, a patient with a bodily or mental complaint will consult a doctor to discover the causes of the complaint. When this happens, personal data on the patient are recorded at the clinic or surgery, such as the patient's first name, surname, date of birth, insurance number, sickness fund, etc., and are retrievably filed as master data, preferably in a data management system. The doctor will make a provisional diagnosis for the patient to be examined, and will obtain a sample, e.g. a blood sample, from the patient for confirmation of this diagnosis based on the doctor's specialist knowledge. A more rigorous analysis of the sample is carried out in a large central laboratory or joint laboratory facility, and in order for this to be done the doctor has to make out a request or order form stipulating the requests, which then goes to the laboratory together with a sample container containing the sample. The analytical results obtained on the sample in the laboratory's diagnostic tests are usually communicated to the doctor orally or in writing. Sample receptacles for interim storage of the sample are provided with a data carrier, in particular a 1D barcode, before or after the sample is taken: and it is by this token that the contents of the receptacle are uniquely matched with the request note provided by the doctor, and with the individual patient. The analytical results are then also assigned to this data carrier. However, a high risk of confusion in matching the request list, sample receptacle and analytical results persists, especially when one bears in mind that these large laboratories process several thousands of such sample receptacles each and every day. For the patient to be treated, the much more serious disadvantage can result that the making of a final diagnosis and the further actions necessary for treatment of the complaint are based solely on the specialist knowledge of one doctor and the doctor may well overlook a possible form of examination of the sample in the laboratory, and situations that are life-threatening for the patient may even be missed altogether.

To counter the risk of mix-ups between samples in the “mass processing” of patients, an extremely wide variety of systems have been proposed in the state of the art.

In one method known from WO 94/22580 A, for instance, labels together with an extremely wide variety of lists, such as request lists of desired parameters, are generated from data on a so-called patient card in conjunction with data from a data memory of a central computer. These labels are stuck directly on to the sample receptacle, and they contain, on the one hand, information data fields with machine-readable data allowing automatic sorting in the laboratory using a distinctive identification code, and on the other hand, a second machine-readable code from which the tests to be carried out on the sample are specified. Detailed information that can be read by humans can be derived from further numeric or alphanumeric character sequences printed on the label. The extremely large number of pieces of information or high data-density yield the disadvantage that a large part of the surface area of the sample receptacle is covered up, and visual inspection of the sample itself may well be difficult, or altogether impossible; and it becomes necessary to provide expensive and sophisticated reading devices which, in order to maintain reading accuracy, must be equipped with special manipulating and orientation mechanisms in the reader station.

All systems known from the state of the art suffer from the drawback that there is a high risk of confusion of the sample containers to be used and/or that a sample container may be wrongfully used for a particular test. The reason for this is the large number of variables or parameters to be determined from samples, e.g. of blood; which also causes a steady rise in the number of “different” sample receptacles. The “differences” lie not so much in the outward form of these sample containers as in their contents, that is to say in the reagents or reagent mixtures presented in them; so incorrect use is an ever-present possibility.

Also known, from DE 19955729 A, is a communication system for co-operation between medical laboratories and medical treatment facilities, in which all participating laboratories and treatment facilities are linked via a data transmission facility with a central laboratory database. The necessary patient and test data are recorded at an input module of a data input device at the treatment facility and, co-ordinated by the central database, are notified to the competent laboratory via a data output device at the competent laboratory. Prebooked orders for tests are activated on arrival of the sample at the laboratory. After the tests have been completed, the test data are transmitted via the data transmission facility to the treatment facility. A drawback is that both the necessary patient data and the test data are transmitted to the laboratory, and this can cause inconsistencies (discrepancies) in data management as a result of the redundant management of patient data by user and laboratory. Moreover, the user of the system must acquire a great amount of specialist knowledge of the numerous different types of sample containers; and the risk of confusion in the use of sample containers cannot be ruled out.

It is the object of the invention to make available a method whereby the reliability of the sample removal and analysis process can be increased. In particular, it is the object of the invention to make available a method whereby an incorrect choice of sample container and/or an erroneously obtained sample can be eliminated.

The object of the invention is realized, independently of each other, by the measure provided in claim 1. The advantage resulting from the characterizing parts of these claims is that the user is able to access a wealth of information, such as for example research results and/or empirical results and/or expert advice, through information catalogues, and, at least, the type of sample container to be used is thereby very easily and unambiguously defined. This moreover saves considerable time for the user as, for example, it removes the need for detailed study of the technical literature regarding sample containers and/or variables to be determined from samples, and manual recording of data, in particular test requests, which may include the variables or parameters to be analysed by the laboratory and/or the analytical requirements.

The object of the invention is also realized independently in claim 2. The advantage accruing from the characterizing part of this claim, besides the advantages described above in relation to claim 1, is that all data necessary for the execution of an order are processed or lumped together into a standard machine-processable order record, and this results in a speeding-up of access in the further processing of these data, as several different data formats or representations of data no longer have to be processed, and different storage media no longer have to be read out from or scanned. A further advantage is that a clear separation is made between the patient data and the order record, and only the order record is released to the commissioned laboratory. The data on the system—for example, the patient data—are therefore recorded, processed, prepared or modified, and retrievably stored in a data management device preferably provided specially for the purpose, in particular a database, only at the user end, and the system is given an identification number through which other data on the system can be accessed: for example, the surname, first name(s), date of birth, sex, status (e.g. pensioner, co-insured spouse or family member, etc.). But only the system's identification number, and not the whole of the data filed under this identification number, is passed to the laboratory by the data record. Hence the only data released to the laboratory or to a central administration system are the order data needed for the further analysis of the sample and enabling the sample or sample container to be positively linked to the system and user. This has the effect of reducing the amount of data at the laboratory or central administration system. The further system data associated with the identification number can be called up by the user by entering the identification number into the input device, such as a PC. The patient data are read into the computer system manually or automatically. When the order record is raised, the container data (preferably distinctive), the distinctive identification number and the test requests are assigned to the serial, unique order number. This unique and unambiguous identification number may be a multi-digit, in particular a four-digit, numerical code, or a 1D or 2D barcode etc., thus reliably avoiding the risk of confusion among the multitude of containers allocated to different order numbers.

A further advantage lies in the measure according to claim 3, whereby the user is informed about the nature of the sample to be taken for a given suspected diagnosis, so enhancing the reliability of the method. Moreover, it represents a considerable time-saving for the user, as, for example, it removes the need for detailed study of technical literature regarding the samples needed for a suspected diagnosis and the variables to be determined, and manual recording of data, in particular test requests, which may include the variables or parameters to be analysed by the laboratory and/or the analytical requirements.

Another possibility is a configuration according to claim 4. The resultant advantage is that the input of the suspected diagnosis and/or of the analysis request, and the output of the information catalogue, can be effected by means of components that are tried and tested and not prone to failure, thus substantially increasing user acceptance of the method. Adoption of a user interface or screen mask affords the further possibility of transferring data from the information catalogue for other purposes, for example for the generation of order records.

The measure in claim 5 affords the advantage that the at least one usable type of sample container is specified to the user, and the putting of a sample into an unsuitable container is thereby effectively prevented. For example, a blood sample might be mistakenly put into a blood sample tube with unsuitable reagents, etc.

The practice of claim 6 is also possible. The resultant advantage is that the nature of the sample to be taken, that is to say the material which the user needs to obtain, is precisely defined to the user, thus enabling the number of erroneously obtained samples to be further reduced.

Proceeding in accordance with claim 7 has the obvious advantage that the user is referred to special sampling conditions, such as for example a specific sampling temperature or a specific sampling point or a specific condition of the system from which the sample is to be drawn, or the user is made aware of the need to take specific factors into consideration.

Adopting the measure of the characterizing part of claim 8 is also possible, and highly advantageous. Since, for almost all samples, the number of variables that can be determined is large, it is very important to specify to the user the normal or recommended variables to be determined for his suspected diagnosis, possibly sparing him from having to make a very time-consuming search of technical literature. In the case of blood, for example, several thousand variables can be determined, and even an expert working in this specialist field will not know precisely and in detail all the possible variables or tests to be performed; and for this reason it is most important to offer to the user a suggestion as to the variables to be determined.

The measure of claim 9 affords the advantage that further information important for sample handling, sample storage, sample processing or sample dispatch, or a probable waiting time for receipt of the results of the analysis, can be divulged to the user.

A configuration according to claim 10 is also possible. The resultant advantage is that the information catalogue can be specially adapted to requirements or capabilities of a particular laboratory, so that requests for analyses which the laboratory is unable to perform are avoided, and the samples to be analysed are dispatched by the user in a form that can be processed by the laboratory.

When a wide range of variables is to be determined, the procedure of claim 11 is advantageous, as, with reference to the variables to be determined, a possible laboratory able to determine all these variables can be identified and divulged or the user can be made aware that not all the variables to be determined can be analysed in a particular laboratory.

Proceeding in accordance with claim 12 has the advantage that unnecessary communication of information is avoided, and the composition of the information catalogue can therefore be compact. For instance, a sample-container manufacturer adopted by the user can be notified to the database; thereafter, wherever possible, the only information catalogue proposed will be one containing sample containers made by this particular manufacturer.

The procedure according to claim 13 describes an advantageous option of transmitting to the database, information on the range of sample containers to be chosen from by the user.

The advantage of proceeding in accordance with claim 14 is that the data suggested by the information catalogue can be supplemented or amended so that the user can very easily notify special wishes or extra requests to the laboratory; so increasing acceptance on the part of the user.

The procedure according to claim 15 affords the advantage that the appointed laboratory can prepare more effectively for the pending order, for example by reserving analytical capacity or preparing analytical stations for the pending order.

The feature of claim 16 is advantageous as by recording the test requests on a data carrier, these test requests continue to be available in machine-readable form.

Especially advantageous configurations of the data carrier and possible arrangements of the data carrier are detailed in claims 17 to 21. Claim 20 describes a configuration of the 2D barcode which has proved in practice to be relatively free from error and easily readable.

The advantage which accrues from the approach outlined in claim 22 is that no additional programme or software is necessary for the input of the suspected diagnosis and/or analysis request, or for the output of the information catalogue. Instead, these data can be input and read out, respectively, on an internet page, in particular an html page; the sole prerequisite is that the computer system has internet access, i.e. an html browser which uses the http mark-up language.

The advantage gained by a method-configuration according to claim 23 is that data in the information catalogue can be read out or presented to the user by audiovisual means.

The procedure according to claim 24 advantageously ensures that data in the information catalogue are up to date as they can be amended by one or more updating agencies.

With a method-configuration according to claim 25 the advantage is gained that the user is made aware of the need to fill several similar sample containers with one sample, and that the sample quantity required by the laboratory is defined.

A configuration according to claim 26 is also possible. The resultant advantage is that standard procedures can be proposed for standard instructions and for simple suspected diagnoses.

The measure according to claim 27 is also advantageous, as mix-ups of sample containers can be virtually eliminated through the user guidance which the container data is able to provide. It also makes it possible to simplify the handling of these sample containers by offering the user menu-driven options for further action. Particularly in the case where the sample container is a blood collection tube, preprogrammed details can be presented to the user by the computer system, and the data input can consist merely of confirmation of these details. The container data necessary for generation of the order record can be read in automatically by the data acquisition device, so that manual input of container data can be eliminated altogether. It is of course also possible, once the container data have been entered, to open the control and/or processing programme from which possible uses for the sample containers can be seen. This has the advantage that it is possible to query, for a particular sample container, the uses for which it is suitable, thereby effectively preventing over-long storage of sample containers which normally have a use-by date.

The measure according to claim 28 is advantageous in that, in the first method-step in the menu-driven programme—in particular a control and/or processing programme—the test requests for the laboratory analysing the at least one sample are input manually via the input device or recorded electronically by the acquisition device, and the container data already read in for the adopted sample container are loaded and the system data previously stored in the database, e.g. patient data such as first name, surname, date of birth, sex, etc. are read out from the database or input manually by the user via the input device or recorded electronically from a chip card by the acquisition device, and at least one order number for this order is preferably raised by the computer system and/or, in a further method-step following completion of the first method-step, the test requests, container data, system data and order number (if allocated) are fully automatically combined or processed—by the computer system—to form a common, standard, machine-processable order record.

The advantage realized by a further development of the method according to claim 29 is that the name of the user or of the person taking the sample is recorded on the order record, which means for example that when certain test variables are detected, particularly in relation to contagious diseases and the like, the user or sample taker can be informed immediately.

The measure according to claim 30 affords the advantage that various data carriers known from the state of the art and proven in practice can be adopted for storage of the order record thereby enabling the reliability of the read and write cycles to be increased.

Advantageous configurations of the data carrier and advantageous locations in which it can be placed are described in claims 31 to 35.

The advantage which follows from the method-configuration according to claim 36 is that the language normally used in Germany for such an application is adopted as the language of interpretation.

An approach as in claim 37 is also possible. This has the advantage that the meaning of all data is established beyond doubt and it is therefore virtually impossible for data whose meaning cannot be unequivocally determined to be present.

A particularly advantageous user guidance facility is described in claim 38. This ensures that no important method-step is overlooked or disregarded, and also that the method-steps are always run through in the same sequence.

Claim 39 describes an advantageous facility for intervention by the user in the carrying out of the method.

A development of the method according to claim 40 is also possible. The resultant advantage is that from, or with, the order record, an order form (which is required e.g. by many sickness funds to be raised as a paper document) is produced, with the further possibility of making the layout of the order form dependent on e.g. the patient's sickness insurance scheme, thus making it very easy to distinguish between order forms and their connection with different sickness insurance schemes, as happens with state-of-the-art order forms raised as paper documents.

Claim 41 describes an advantageous facility for the initial and subsequent identification of patients, subsequent identification of patients being explained in claim 41.

Advantageous configurations of order numbers are described in claims 43 and 44.

Also possible is a procedure according to claim 45. The resultant advantage is that a check can be made to ascertain whether advance information has been electronically received on incoming order records, and if so, whether preparations have been made for the execution of the order, such as for example the adaptation of analytical apparatus for the execution of the order.

Through a configuration according to claim 46 the advantage is gained that the time spent on initial sorting and allocation to analytical stations in the laboratory can be reduced.

By a development of the method according to claim 47 the advantage is gained that data from the laboratory, such as analytical results for example, are transmitted very quickly to the user, and customer acceptance of the method according to the invention can thereby be further increased.

Feasible and advantageous configuration options for the data identifying, and subsequently recognizing, the user or the person who takes the sample are described by claims 48 and 49.

Advantageous uses of the method are described in claims 50 to 52.

The invention will now be described in detail with the aid of embodiments shown in the drawings.

In the drawings:

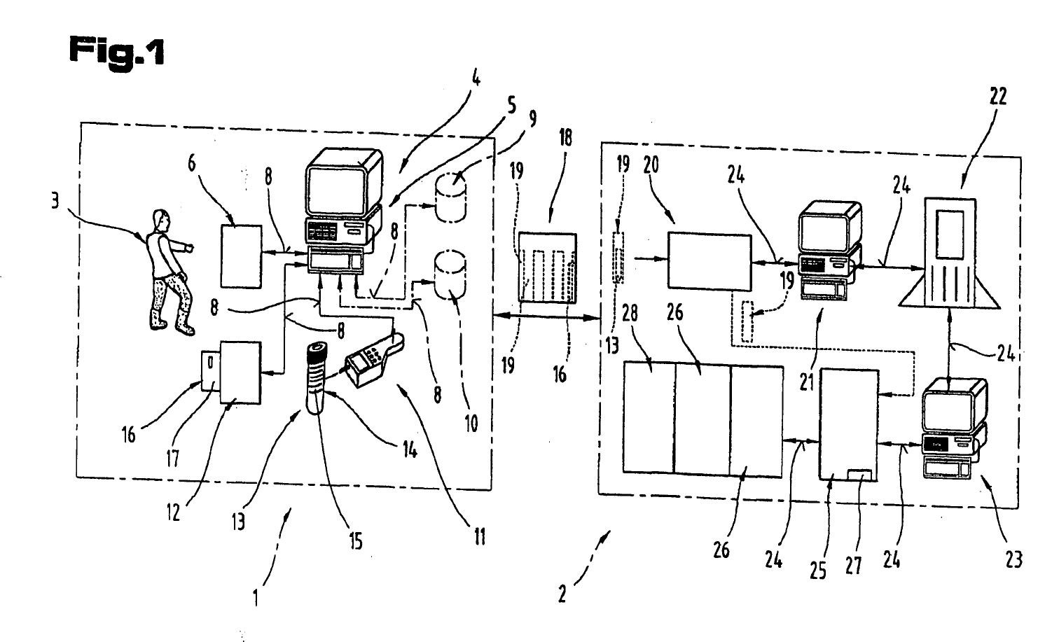

FIG. 1 shows a sample management system for a sample to be handled between a user and a laboratory, with physical data-transmission, in the form of a flow diagram;

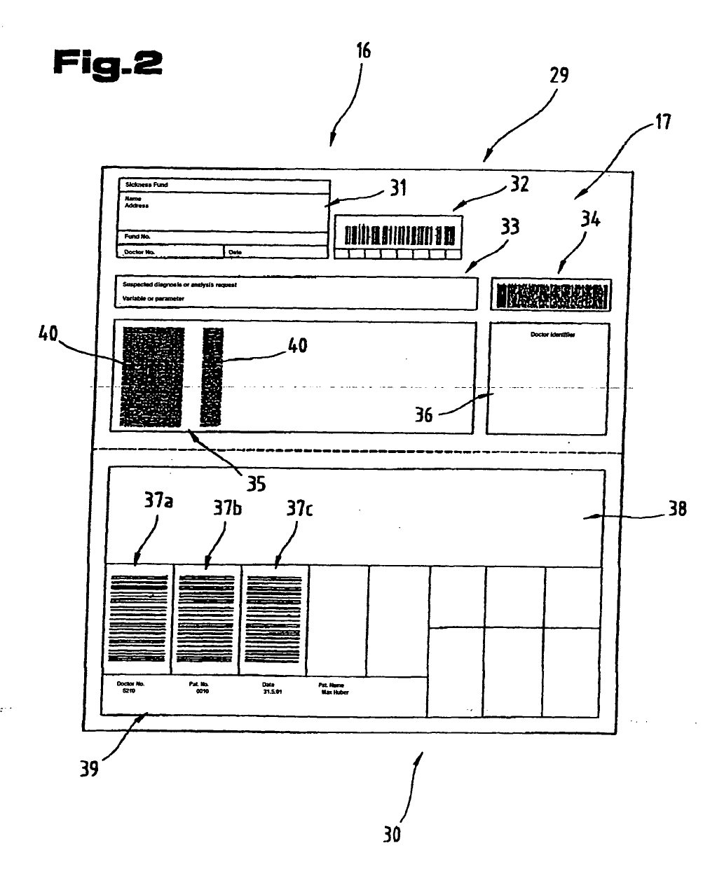

FIG. 2 shows schematically a data carrier, in particular an order form;

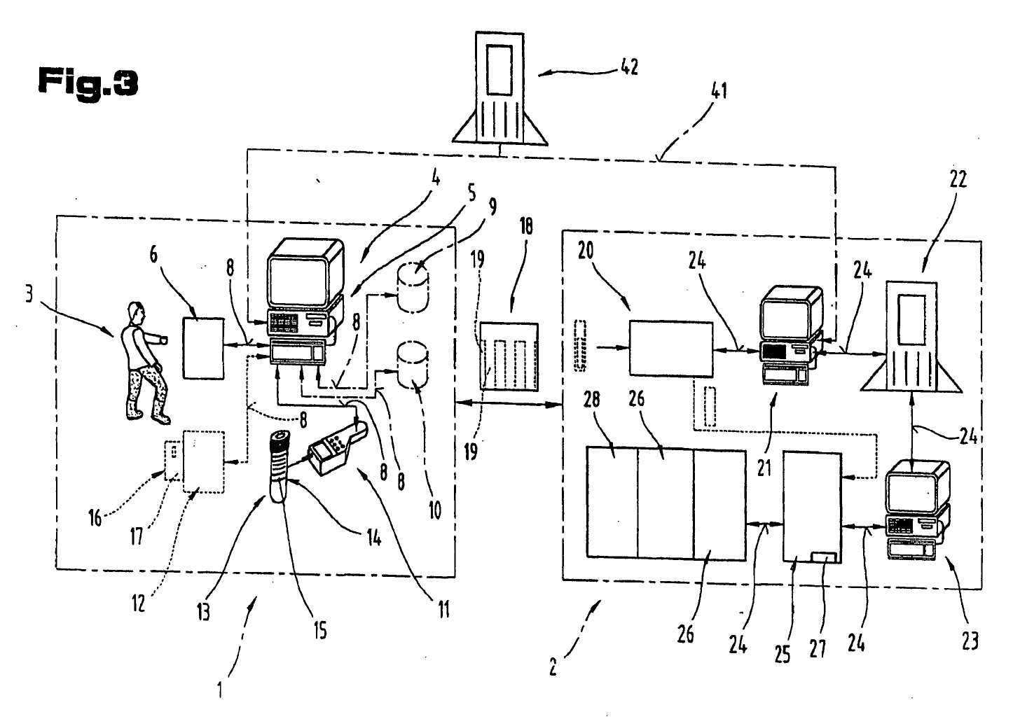

FIG. 3 shows a sample management system for a sample to be handled between a user and laboratory, with in particular Internet-based data-transmission, in the form of a flow diagram.

Let it be emphasized from the outset that, in the variously described embodiments, similar parts are given the same reference numbers and the same component designations, the disclosures contained in the description as a whole being applicable mutatis mutandis to similar parts with the same reference numbers and/or the same component designations. Also: indications of position adopted in the description, such as above, below, laterally, etc. refer to the figure presently described, and should be transposed as appropriate where there is a change of orientation. Furthermore, individual features or combinations of features of the various embodiments illustrated and described may constitute inventive solutions, or solutions according to the invention, in their own right.

FIG. 1 shows in flow chart form a sample management system, from the recording of a laboratory order by the user 1—in particular a doctor's surgery, clinic, or home carer, or research centre for soil ecology—through to the analysis of samples in a laboratory 2. Samples are taken from a system, in particular a biological and/or chemical and/or industrial system, e.g. a human being, an animal, or a chemical or industrial installation. For a better understanding of the invention, the invention will be described with reference to the embodiments which follow, though it is pointed out from the outset that these are to be understood as non-limitative of the extent of protection. In particular, it is assumed that the user 1 is a person, in particular a doctor. The user 1 can of course be personified by an ecologist etc. Patient data on a patient 3 consulting the user 1, e.g. a doctor, are input/recorded/transmitted via an input and/or output device 5 of a computer system 4, e.g. a PC. The input and/or output device 5 is in the form of a PC, and possesses means for manual and/or automatic input of patient-data, and a storage device or data management device for storing or managing same, and also means for outputting at least the patient data retrievably filed in the storage device or data management device. The input means may take the form of an operator interface, e.g. a touch-screen, keyboard, etc., or, as shown here, a terminal 6, in particular a reading device, e.g. a card reader, transponder reader, barcode reader, scanner, etc. The terminal 6 may also (as is not shown here) be an integral part of the input and/or output device 5. This terminal 6 is an independent unit, and is located for example in the reception area of a doctor's surgery; and the patient's identification number and/or personal details, or patient data, which have been read out from a patient card by means of the terminal 6 are transmitted to the input and/or output device 5 via a data line 8. The patient's personal details, or patient data, consist of e.g. surname, first name(s), date of birth, sex, race, status (e.g. pensioner, co-insured spouse or family member, etc.); and the patient's identification number reflects e.g. the insurer, social security number, etc. Patient data recorded for the first time are filed in the data management device 9 in such a way that the patient 3 is uniquely identified solely by inputting the patient identification number. The data processing device 9 shown in chain-dotted lines in FIG. 1 and linked for data transmission purposes by the connecting line 8 is formed by a doctor's (or doctors') master database.

Otherwise the terminal 6 may also have biometric sensors which checks the authenticity of the patient 3 by accessing data or features of the patient 3 identified by the patient identification number which have been retrievably filed in the storage or data management device, so that a positive identification of the patient can easily be performed within the computer system 4. All that is required is e.g. to read out an insurance number from the patient card, whereupon the patient's further personal details assigned to this insurance number and fetched from the storage and/or data management device are automatically displayed on the output device 5. By adopting this unique assignment of e.g. the insurance number to the patient's data filed in the data management device 9, the time taken to record the patient data can be considerably reduced, since the personal data of the patient 3 need only be registered on a single occasion and need not be reentered in the data management device 9 unless there is a change in the personal data. Identity data on the doctor, e.g. first name(s) and surname, sickness fund number, etc., are likewise stored, or retrievably filed, in the storage device or data; processing device 9 or doctor's master database.

As suggested by chain-dotted lines in FIG. 1, a further data management device 10, in particular a database (metadatabase), may be provided. It is connected by the data line 8 to the input and/or output device 5 for transmission of the data read out from the data management device 10 to the input and/or output device 5. The data management devices 9, 10 form an integral component of the computer system 4 in this embodiment.

In this further data management device 10 or further database, which is provided in the user domain 1, are retrievably filed or stored a large number of suspected diagnoses for samples to be obtained e.g. by the doctor, in particular clinical pictures, or information catalogues germane or assigned to requests for analysis, so that following the input of at least one suspected diagnosis, e.g. cardiac infarction, and/or of at least one analysis request, e.g. for a large blood count, the computer system 4 automatically establishes a connection to the database and finds the information catalogue assigned to the suspected diagnosis and/or to the analysis request, and this is then offered, or amendably presented, via the output device 5 of the computer system 4. On the basis of the entered suspected diagnosis and/or analysis request, the at least one type of sample which the information catalogue suggests should be taken, e.g. a blood sample, is output. On this sample, variables or parameters such as e.g. cholesterol levels will need to be analysed by the laboratory 2. To facilitate processing of the input of the suspected diagnosis and/or analysis request, predefinable identifiers such as code numbers etc. may be allocated to these. Input of the suspected diagnosis and/or analysis request is effected via at least one user interface, e.g. a screen mask, displayed at the input and/or output device 5. What kind of data are included in the information catalogue, and how the individual method-steps can be performed, will be explained in the following description. The invention provides that the patient data are manually or automatically transferred into a computer system 4; test requests for the laboratory 2 are generated thus: an information catalogue which is germane to an analysis request and/or suspected diagnosis and whose data are supplemented or amended if required, is suggested, and the test requests are generated from at least some of the supplemented or amended data; and container data on at least one sample container 13 specified for the sample are defined; and a common, standard, machine-processable order record is generated by the computer system 4 from the totality of all these data.

Test requests for the laboratory 2 are generated from the information catalogues suggested by the computer system 4 and/or read out from the database 10, and/or from data from the information catalogue supplemented or amended by the doctor. These test requirements are combined in the computer system 4 with the system identification number, e.g. the patient's identification number, the research centre identification number, the clinic identification number, etc., and with container data detected by an acquisition device 11 and/or with user-specific identification data, e.g. doctor's location, department in a hospital, doctor's name and address and/or identification data on the person who takes the sample, etc., to form at least one order record, which are displayed by the output device 5 on the screen of a PC or printed out by a printer device 12 and/or stored or retrievably recorded on a physical data carrier (not shown in the drawing), in particular a magnetic stripe and/or chip and/or transponder and/or rewritable plastic card or floppy disk, CD, order form. This test request and/or the order record are recorded or stored on a data carrier, in particular a physical data carrier, e.g. an order form etc., or are transmitted electronically via a global or local data network, e.g. the Internet or an intranet, as shown in FIG. 3. These can be transmitted to the appointed laboratory 2 as advance information, independently of the sample container 13.

A sample container 13 holding the sample carries, inseparably arranged on it, at least one data carrier 14 with container data unmistakably identifying the container. In this first example embodiment, the method according to the invention may be used for a sample-collecting receptacle, e.g. a blood collection tube. Such receptacles usually consist of a single-layer or multilayer receptacle-body, preferably of plastic material, and, if they are evacuated so that the sample, in this case blood, can be drawn up automatically, are fitted with a gastight cap. This cap usually comprises a self-sealing septum that can be pierced with a cannula. This sample receptacle may of course also take the form of e.g. a capillary blood collection system, faecal receptacle, swab specimen transport tube, blood-bag, etc.

Owing to the ever-growing number of variables or parameters to be determined from samples, e.g. blood, the diversity of sample containers 13, e.g. blood collection tubes, is also constantly increasing. The differences lie not so much in the outward shape of these sample containers 13, e.g. blood collection tubes, as in their contents, that is to say, the reagents or reagent mixtures presented in them. For example, additional coagulants (or, contrariwise, anticoagulants) or stabilizing or lysis-inducing reagents may be contained in the blood collection tube.

To eliminate this problem of confusion between sample containers 13 and/or incorrect use of a sample container 13 for a given test, in accordance with the invention at least one information catalogue is presented to the user 1, from which he can be prompted to obtain or call up information. Depending on the suspected diagnosis and/or analysis request entered, the user 1, in particular a doctor, is informed of the necessary type of sample to be taken and/or the appropriate sample container 13 for this suspected diagnosis and/or analysis request. Of course, it is possible to arrange matters so that that only those sample containers 13 which the user 1 actually uses are specified, by recording in the information catalogue the sample containers 13 e.g. of a particular manufacturer that the user uses. The sample containers 13 are usually picked up manually from a store. It is of course possible to provide at least one automatic dispenser on the user's premises which also comprises the acquisition device 11, in particular a data reader, e.g. a scanner, barcode reader, or CCD camera, which automatically identifies the sample container 13 on delivery. In this case, an instant comparison is made between the sample container 13 suggested by the information catalogue and the sample container 13 dispensed, so that incorrect use is ruled out. When the suggested sample container 13 is used, the acquisition device 11 of the computer system 4 detects the container data of the sample container 13 from the data carrier 14 (which preferably has already been placed on the sample container 13 by the manufacturer), electronically records or reads these container data and transmits them to the computer system 4, whereupon, once the sample container 13 has been given a unique identification, a menu-driven programme or user interface, e.g. an html page or a control and/or processing programme, is automatically activated in which the user 1 is requested to input further method-steps such as input of test request, order number, identification number of the system, which are then combined in an order record which will be described in detail later.

As can be seen from FIG. 1, this comparison between the suggested sample container 13 and the (manually removed) sample container 13 is made by the acquisition device 11. If the data carriers 14 have already been put in place by the manufacturer, container data such as the code or type of sample container 13 and/or its place of manufacture and/or use-by date, etc., can be read out at the same time. If for example the use-by date is imminent or is reached or has been exceeded, advice is given automatically via the in and/or out device 5 in the programme or user interface, before this sample container 13 is used, that this sample container 13 can no longer be used or, where an at least partly automated dispenser is used, the “time-expired” sample container 13 is dumped in a waste bin.

The data carrier 14, which may contain the order record if required, but conveniently only contains at least the machine-readable container data, is, in the present embodiment, configured as a one-dimensional 1D barcode 15 inseparably placed on the sample container 13. Here the data carrier 14 is configured as an adhesive barcode label. Data in alphanumeric form can of course be used instead, or additionally placed on the data carrier 14. Preferably, the 1D barcode 14 forms a multi-digit code identifying the sample container 13 and also the container contents. This code may for example consist of 12 digits; and it is possible for some of these 12 digits to record fixedly on the sample container 13 the individual batch forming the container data and also manufacturing data, for example the use-by date, place of manufacture and size of the sample container 13 as well as the date of manufacture (showing year and month of manufacture), a doctor number or user-specific identification data and/or test requests etc., the last two also being recordable on the spot as variable data.

This data carrier 14 can of course also be formed by a transponder known from the state of the art, with a transceiver and a storage unit, or by an optionally rewritable memory chip. For this embodiment of the sample container 13, the data carrier 14 may be configured for example as a film chip. Such film chips, which are also available in transponder technology, can be obtained as bulk film in the form of film labels.

The data carrier 14 may also be constituted by the material of the sample container 13 itself. In this case the data are written directly into the material. The sample container 13 is made of plastic material such as e.g. PE, PP, PET, PAN, PS or the like. Thus it is possible for example not only to produce sample containers 13 from light-modifiable polymers (for example by adding photochromic reagents which e.g. are cured in particular by ultraviolet light thus producing a different optical behaviour of the material), but also to produce sample containers 13 on which the inscription can be made by the application of heat. It is known that specific reagents or polymers change their chemical and/or physical properties under thermal stress, and that this change can be fixed. By highly selective heating of specific areas on the sample container 13, a binary code, or the one-dimensional barcode, can be imparted by this method. The inscription, or rather, the material of the sample container 13, can also be modified by additives so that repeated inscription is possible, and so that data, in particular container data etc., can also be erased. The data are recorded on or inside or within the sample container 13. Of course, the possibility also exists of making the data carrier 16 which is to be physically transmitted and which bears the order record, from plastic material such that this data carrier 16 changes its properties when heated, so that data can be written or erased. Thus, repeated inscription of the data carrier 16 is possible. This principle has already been described for the data carrier 14, and are therefore adopted. A relevant device for inscribing or erasing data is known for example from EP 0431155 B1.

Conveniently, the at least one data carrier 14, in particular the 1D barcode 15, is affixed or arranged on the sample container 13 by the manufacturer in the form of a label or a memory module such as a chip, so that the user 1 ultimately has no further manipulation to perform, apart from scanning. The data carrier 14 is sufficient for the identification of the sample container 13 and/or of its contents i.e. the sample. On the other hand, the data carrier 14 may also take the form of an engraving. This variant has the advantage that the 1D barcode 15 is inseparably joined to the sample container 13, which is not the case with labels that can be peeled off.

However, instead of barcodes, it is possible to use alphanumeric data records that can be read by the human eye. Reader devices for the transmission of data to the input and/or output device 5 are also available for data presented in this form. Such alphanumeric data may of course be coded.

These data on the sample container 13 are read out contactlessly by the acquisition device 11, in particular a hand scanner, and transmitted to the input and/or output device 5 for further processing and generation of the order record. The acquisition device 11 and the input and/or output device 5 may be permanently linked to each other by cable for this transmission of data, though versions with infra-red interfaces are also feasible. For the transmission of container data from the container 13 to the acquisition unit 11, a contactless transmission, e.g. again by means of an infra-red interface, is feasible. Instead of the hand scanner, a fixedly installed scanning device or a memory chip reader etc. (not shown in FIG. 1) into which the container 13 is inserted, may be used.

As briefly stated above, the order record if necessary includes user-specific, in particular doctor-specific, identification data, which are preferably recorded and stored on a once-only basis; and biometric features, in particular a fingerprint and or an iris method and/or facial shape are used for subsequent recognition of the doctor, whereupon the corresponding identification data for the doctor are automatically read out and made available or utilized for the generation of the order record. The biometric features are preferably stored in the storage device or doctor's master database, and are preferably write-protected.

Once the suspected diagnosis and/or analysis request has been entered, data on the type of sample that needs to be taken and/or at least one sample container 13 that needs to be removed for the suspected diagnosis and/or analysis request are presented automatically by the computer system 4, and the suggested sample(s) and/or sample container(s) 13 is (or are) removed. The at least one suggested sample container 13 from the information catalogue is made ready and is positively and unmistakably identified by the acquisition device 11, and the container data acquired are combined with at least the system identification number, e.g. patient identification number, and the test requests raised and/or user-specific, in particular doctor-specific, data, as at least one order record. During or before or after the removal of the sample, e.g. a blood sample, a data carrier 16 uniquely related to this order, in particular an order form 17, is produced by the printer device 12. The at least one sample removed is stored in at least one sample container 13 for a predetermined period and sent to the laboratory 2 for the analysis stipulated by the test requests.

For this purpose, a mobile container 18 is provided on the part of the user 1 to hold a number of mounting device 18 for, preferably, several sample containers 13. The order form 17 for each order (possibly covering several sample containers 13) is enclosed, so that the samples and sample containers 13 in the mounting device 19 delivered to the laboratory 2 are unambiguously identified. The box-like container 18 has several compartments so that a plurality of mounting devices 19 can be held and secured, and at least one compartment in which the order forms 17 relating to the sample containers 13 can be inserted. In this specific embodiment the mounting device 19 is constructed as a so-called analysis rack. Such racks are already known from the state of the art. In the simplest case, such racks are configured as specimen stands. The multi-functionally configured racks are moreover configured to hold sample containers 13 of different dimensions, thus allowing high flexibility in the physical transmission of a number of different sample containers 13 from the user 1 to the laboratory 2. It is expedient to also provide the mounting device 19 with at least one data carrier 14 (which is not shown) consisting, as described above, of a 1D or 2D barcode, a transponder, a film chip, the material of the device 19, an engraving, etc. The order record, in particular the container data, system identification number, test requests, doctor's identification data, etc., can of course also be stored or retrievably recorded in the data carrier 14 provided on the mounting device 19.

The possible layout and data content of the order form 17 will be described in detail with reference to FIG. 2.

As is also clear from FIG. 1, a spatial separation exists between the user 1 (e.g. doctor's surgery) and the laboratory 2 between which the physical transmission (e.g. delivery service) of the sample containers 13 and/or data (e.g. on the order form 17) takes place. It is of course possible to send to the laboratory 2 with the container 18 not only order forms 17 as described above, but also other physical data carriers 16 such as e.g. magnetic stripes and/or memory chips and/or film chips and/or transponders and/or floppy disks and/or CD-ROMs.

By means of the data on the physical data carrier 16, in particular the order form 17, all data needed by the laboratory are dispatched together with the sample containers 13; and these data can be matched with the individual sample container 13 by virtue of the fact that the data are recorded on the data carrier 14 of the sample container 13.

The sample containers 13 and/or mounting devices 19 sent to the laboratory 2 are checked upon receipt by comparing the container data on the sample containers 13 with the order records on the physical data carrier 16 or on the mounting devices 19 or on the sample container 13. This incoming check is conveniently made at an order recording point 20 at which the transmitted physical data carriers 16 are processed accordingly. The order recording point 20 may comprise for example a scanner for the order forms 17 and/or a barcode reader and/or a chip reader and/or a CDROM drive unit etc. with corresponding software modules for further processing of the transmitted data into a data format suitable for an input and/or output device 21. The data read out from the physical data carrier 16 are stored or retrievably filed together with the container data of an order in a laboratory information system 22.

For this purpose the laboratory information system 22 also has a database to manage the data associated with the order, in particular the order records and any container data separate from the order records. A system control centre 23 is linked by a data line 24 to the laboratory information system 22 for transmission of data filed in the latter. The primary advantage of the separation between the laboratory information system 22 and the system control centre 23 is that the only operations conducted in the laboratory information system 22 are those of managing the data, in particular the order records and any container data separate from the order records; while the system control centre 23 is exclusively concerned with driving at least one sorting unit 25 and individual analytical stations 26 in an optimized manner. For the acquisition of data through the system—from the incoming containers 18 or mounting devices 19 to the analysis of the individual sample in the analytical stations(s) 26—the order recording point 20, input and/or output device 21, laboratory information system 22, system control centre 23, sorting unit 25 and analytical stations 26 are linked up for data transfer purposes by data lines 24 forming a local data network.

The physical transfer of samples or sample containers 13 (singly, or grouped as an order) is shown in broken lines. Following receipt of the containers 18 in the laboratory 2, the individual mounting devices 19 are removed and passed to the sorting unit 25. The sorting unit 25 comprises at least one conveyor system (not shown in FIG. 1) such as e.g. a belt conveyor or the like for mounting devices 19 or individual sample containers 13, and at least one acquisition device 27, e.g. photoelectric sensors, CCD camera, sensors etc., for reading the data recorded, preferably in machine-readable form, in the data carrier 14 and/or data carrier 16 on the sample container 13, in particular the container data and/or the data from the data carrier 14 of the mounting devices 19. At least the container data obtained by the acquisition device 27 are read into the laboratory information system 22, the identified sample container 13 is automatically matched with the transmitted data relating to test requests presently in the system control centre 23, and the mounting device 19, or sample container 13, is automatically allocated to the individual analytical station 26.

Hence only the test requests necessary for the analysis of the samples are transmitted to the sorting unit 25 for selective forwarding of the samples for analysis to the individual analytical stations 26; so that repeated storage of data within the laboratory 2 is no longer necessary. On the basis of the data recorded in the data carrier 14 on the sample container 13 and/or in the mounting device 19 and/or the physical data carrier 16, e.g. the test requests stipulated by the doctor, the sample containers 13 are split up among individual analytical stations 26 for e.g. luminescence readings, PCR, pH determination, etc. Such analytical apparatuses for determining individual sample-variables are known from the state of the art and already in use in the various laboratories, so that a further enumeration here is not necessary.

It would of course be possible to provide just the system control centre 23, containing a storage device of corresponding storage capacity or an integrated database.

Within the laboratory 2 itself, several functions are covered by the sorting unit 25, e.g. in addition to sorting and/or positioning and/or orientating and/or registering the sample containers 13, it may also carry out a quality inspection e.g. of the filling level, colour and opacity of the sample, and layering e.g. where serum tubes are used, possibly including a check of the colour and/or opacity of the individual layers, removal of the stopper or the opening-up in general of the sample container 13, placing on the analysis racks or the like, discharge of rejects. In order to accomplish this, the sorting unit 25 is preferably of modular construction so that it can be adapted to the individual requirements of the laboratory 2. Preferably, the user 1 is informed by the laboratory 2 through the information catalogue as to the necessary quantity, e.g. the volume of blood, to be obtained so that the analyses called for in the test request can be carried out sequentially. If, however, insufficient sample material is available for a test request e.g. following loss of a sample container 13, the doctor is automatically notified through a written or electronic or oral message.

After the analysis is completed, the analytical results are made available in data form via the data link between the analytical stations 26 and the system control centre 23 and/or laboratory information system 22. These analytical results are preferably sent to the user 1, i.e. the doctor in the present example, on a physical data carrier 16, e.g. a CD-ROM, floppy disk, analytical results form, fax confirmation, etc. The transfer of the container 17 from the user 1 to the laboratory 2, and of the physical data carrier 16 with the results of the at least one sample analysis performed, is preferably effected by post. The physical data carrier 16 received by the user 1 is read by the acquisition device 11 and the analytical results are displayed, e.g. on the screen, at the output device 5, or printed out on the printer device 16 for the issue of a report.

Otherwise, the possibility also exists (though is not shown here), if the need arises, of recording or storing the analytical results in the data carrier 14 or in a data memory on the mounting device 19 and/or on the container 18 forming this data carrier 14, so that they can be read out by the user 1 via the acquisition device 11. After the analysis of the samples is complete, they can be fed to an archive station 28, either separately or with the mounting devices 18, for further storage (unless they are to be simply discarded). Archiving the samples may afford the added advantage that further sample-taking is no longer necessary. This archive facility 28 may possess suitable installations such as e.g. a cooling system, temperature control, etc.

Similarly, the analytical results are stored, preferably in the laboratory information system 22, for as long as the samples are kept, and after the latter have been discarded are preferably automatically deleted. Should the need arise, the samples in interim storage in the archive facility 28 may be subjected to a repeat analysis or check analysis. A request for such a repeat analysis of a sample can be initiated by the user 1 by sending to the laboratory 2 a further physical data carrier 16, e.g. an order form 17, referring to this order.

The laboratory information system 22 is also able to process urgent jobs, for example when samples have to be dealt with on a priority basis. In this case, the sorting unit 25 and analytical station 26 (and possibly the archive facility 28) are mobilized accordingly by means of the system control centre 23.

To maintain or update the information catalogues filed with the user 1 in the database 10, the possibility exists of supplying the user 1 at predetermined intervals, e.g. every six months, with data carriers such as a CD or floppy disk for an update of the information catalogues and/or application software, especially the user interface.

The physical data carrier 16, or the order form 17 which embodies it, is shown schematically in FIG. 2. This physical data carrier 16 is, as shown in FIG. 1, raised by means of the printer device 12 of the computer system 4, from a multitude of data.

The order form 17 generated by the user 1 is preferably in DIN A4 format, and has two portions 29, 30 joined by a perforation which is represented in FIG. 2 by the broken line. These two portions 29, 30 have a large number of data fields 31 to 39 which, as explained with reference to FIG. 1, contain the patient data, in particular the patients personal details and the patient identification number, the identification data of the user 1, in particular the panel doctor number or identification data of the person who takes the sample, and the test request data and container data, and also if required an order number and the date of sample collection, all in machine-readable form, in particular as graphic data, e.g. in 1D or 2D barcode form, and/or in alphanumeric form. It will be seen that in this example, data fields 31 to 40 are provided, each with its distinctive storage capacity. Individual data fields 31 to 40 may contain data in both machine-readable and alphanumeric form, as is suggested in data field 32.

To facilitate subsequent handling of the sample within a sample management system, data including all data required by the laboratory 2 for handling and further processing of the sample are dispatched together with the sample container 13; the sample container 13 can be matched to an order through the record of container data in the 1D barcode 15. The data comprise the system identification number, test requests, container data, order number, and (if required) identification data on the doctor and (if required) the date of sample collection, and are retrievably recorded as a standard machine-processable order record in a single data field 35 as a graphic data record, in particular in the form of a 2D barcode. All these data are presented as a standardized order record and therefore in the same data format and in the same form of presentation and as the same interpretation language, and they are retrievably recorded in a 2D barcode. The order record is split up into a number of separate data blocks 40 of defined memory size. PDF-417 is used for the 2D barcode. The whole of the data constituting the order record is resolved into a number of data blocks 40, the memory size of one data block lying in the range between 100 bytes and 1300 bytes, and in particular between 600 and 1100 bytes, e.g. 950 bytes. Assuming one data block 40 has e.g. a 950 byte storage capacity, the entire order record may have up to six, and in particular four, data blocks 40 each of 950 bytes. The storage capacity of the data block 40 may of course be smaller, for example half as great i.e. 475 bytes. Hence the possible memory size of the order record is obtained by multiplying the number of individual data blocks 40 by the storage capacity of each individual data block 40. Basically, it can be assumed that 1 byte corresponds to one character.

In order to make an incoming visual check, or for that matter any visual checks, of individual data of the sample containers 13 sent to the laboratory 2—as shown in FIG. 1—or during transport of the sample containers 13, the data can be read out from the container data by means of a hand terminal, e.g. a palm-size data terminal, which can be supplied with the package, and compared with the alphanumeric data on the order form 17. The container data, e.g. the 1D barcode, include the order number by which data such as sample handling and sample dispatch can be retrieved.

As illustrated in FIG. 2, patient data and/or user identification data, insurer, sickness fund, and date of sample collection are recorded in alphanumeric form in the data field 31. Similarly, the analysis requests and/or suspected diagnosis and/or test requests including sample variables or parameters to be determined are, if required, shown in alphanumeric form in a separate data field 33.

In a further data field 32, a serial, unique order number issued by the user 1 can be retrievably recorded in machine-readable and/or alphanumeric form. Hence this data field 32 enables an unambiguous match to be made between the order form 17 and the sample stored in at least one sample container 13, and/or sample containers 13 and/or the patient and/or the order.

A further data field 38 is provided in another portion 30 of the order form 17 for any remarks in connection with the request, for example additional information regarding sample handling, e.g. sterile conditions, or sample storage temperatures. These remarks are preferably also presented in alphanumeric form, possibly coded if this is necessary on data protection grounds. Here it should be pointed out that all data presented in alphanumeric form are readable with state-of-the-art scanners with the appropriate software, and can be processed in accordance with existing algorithms for data processing programmes.

If the sample containers 13 available to the user 1, e.g. to the doctor, are unprovided with a data carrier 14 for the distinctive identification of the sample containers 13, then at least one data carrier 14 matched to the order, or at least one barcode label which constitutes the data carrier 14 and can be stuck on to the sample container 13, is produced. According to this embodiment, a number of barcode labels, e.g. three, can be automatically raised with the aid of the information catalogues, and printed out, by the computer system 4, as shown in FIG. 1. These barcode labels are merely used to provide distinctive and unique identification of at least one sample container 13 or to identify several sample containers 13 belonging to one analysis request, e.g. a request for a large blood count, in which case at least some of the places of the one-dimensional barcode may contain the order number, so that several sample containers 13 provided with these barcode labels can be positively matched with an order. Of course, an order with a given order number may cover two, three, or more sample containers 13. The 1D barcode labels raised for an order are generated in the data fields 37a, 37b and 37c.

If required, patient data such as identification data of the user 1, date of sample collection, patient data, etc. can be printed out in alphanumeric form in yet another dedicated data field 39. This data field 39 is configured as an adhesive label and can be stuck in a doctor's book, e.g. for record purposes.

The number of data fields 37a to 37c; 39 can of course be varied by varying the size and hence the storage capacity, which is adapted to the necessary data content of the data fields 37a to 27c, 39.

The unique, distinctive order number (in particular a serial number) issued by the user 1 can of course also form part of the order record contained in data field 35. The additional information in data field 38 may also form part of the order record.

In FIG. 3 the sample removal system with the user 1 and the laboratory 2 which is to analyse the sample are shown in highly simplified and schematic form. So that the invention can be readily understood, it is again assumed that the user 1 in this embodiment is a doctor. This sample management system can of course also be used in other fields, such as analysis of biological samples in soil ecology, etc. As already described with reference to FIG. 1, in the user domain 1 there is a computer system 4 which comprises at least one input and/or output device 5, at least one means for manual and/or automatic input of patient data and at least one storage device or at least one data management device 9 for storing or managing patient data, and also means for outputting at least the patient data retrievably filed in the storage device or in the data management device. The patient data, and if required, identification data on the doctor, are likewise retrievably filed in the data management device 9 or doctor's master database.

The means for inputting patient data consisting of patient identification number and/or patient's personal details and the identification data on the doctor can be input by the means already described above. The computer system 4 is also provided with at least one acquisition device 11, in particular a hand scanner, for readout of container data recorded in data carriers 14 inseparably placed on the sample container 13. The data picked up by the acquisition device 11 are transmitted, as shown, via a data line 8, in particular a cable; though variants using infra-red interfaces are also feasible. Such acquisition devices 11 are known from the state of the art; all such systems can, therefore, be used for the method according to the invention. The data management device 10, in particular a database, which is also linked with the computer system 4, contains the data retrievably filed in the information catalogues for the analysis requests and/or suspected diagnosis, such as data on the material to be removed and/or sample containers 13 and/or special conditions for sample collection and/or the at least one sample variable to be determined, etc. If required, an output device 5 in the form of the printer device 12 shown in broken lines can be provided, for example to enable a continuous record to be-kept, for example in a doctor's book. If so, a data carrier 16, in particular a log, can be issued, containing data in alphanumeric or machine-readable form, so affording a physical means independent of the computer system 4 of reviewing examinations carried out, patient data, etc.

As has also already been described in detail with reference to FIG. 1, the laboratory 2 comprises at least one order recording point 20, at least one input and/or output device 21, at least one laboratory information system 22, at least one system control centre 23, at least one sorting unit 25 with an acquisition device 27, at least one analytical station 26, and at least one archive facility 28. The sample containers 13, or samples and mounting devices 19, coming into the laboratory 2 enter the sample circuit already described in detail with reference to FIG. 1, which, to avoid repetition, will not be described in detail with reference to this figure; the description already given by way of example also applies to this figure.

FIG. 3 differs fundamentally from FIG. 1 only in the manner in which the order record—composed of the system identification number, container data and test requests and (if required) identification data on the user 1 (e.g. the doctor) and/or identification data on the person taking the sample—is transmitted: it is sent electronically to the input and/or output device 21 or laboratory information system 22 of the laboratory 2 via a global data network 41, e.g. Internet or intranet. Hence the transfer or transmittal of the sample containers 13 between the user 1 and the laboratory 2 is independent of the transmission of data. As suggested in the drawing by a two-way arrow between the input and/or output device 5 and the input and/or output device 21 or laboratory information system 22, the user 1 and the laboratory 2 are able to exchange data e.g. order records and analytical results, and/or to communicate with each other, so that this direct communication can save the trouble and expense of issuing physical data carriers 16. The order records supplied by the user 1 to the laboratory 2 are edited in the input and/or output device 21 so that a sample run starting from the receipt of the samples and proceeding through to the storage of the samples can take place; or these order records are temporarily retrievably stored in the laboratory information system 22, or in its database. The order records filed in the database can be retrieved at any time by the input and/or output device 21 and edited accordingly for further processing. Since the order records, which may also contain an order number in the form of machine-readable data, are recorded in the file, an incoming check can be made in the laboratory 2 by a comparison of the data retrievably filed in the database, e.g. the order number, with the data contained in the data carrier 14 placed in the sample container 13 and/or on the mounting device 18.

After the sample has been taken, and, if required, the sample container 13 has been inscribed and/or arranged, an additional data carrier 16, in particular an order form 17, can, if necessary, be raised automatically for dispatch of same to the laboratory 2 conducting the analysis. This additional data carrier 16 can carry predetermined data, e.g. order number, patient data, etc., for the check of the sample container 13 on arrival (or for any check made during transport).

When the positive incoming check is made, i.e. when the data supplied by the doctor via the data network 41 and/or data carrier 16, e.g. order number and/or container data etc., are matched with those on the sample container 13 and/or mounting device 19 and/or data carrier 16, they can be entered by the laboratory 2 in the input and/or output device 21 or, for management of these data, in the laboratory information system 22. Obviously, at least one reading device, e.g. a laser, scanner, barcode laser, etc, will also be installed in the laboratory 2 for this purpose, making it possible both for the container data present on the sample holder 13 to be accepted automatically and, where the data provided by the user 1 has been sent via the data network 41, for an automatic incoming check to be performed. This provides a positive matching of the sample or sample container(s) 13 with the order records, and, in particular, with the patient 3.

In the event of a discrepancy between at least the order number of the order record and the container data associated therewith, outsorting of samples that are not positively identifiable can be done at the receiving point, and the delinquent samples can be returned in due course to the doctor's surgery.

Samples are automatically processed in the actual laboratory 2 on the basis of the order data in the input and/or output device 21 or in the laboratory information system 22. To that end, in accordance with defined sorting criteria, e.g. the variables or parameters to be determined, they may be fed, preferably in timed cycles, to the individual analytical stations 26 or to the archive facility 28.

The variables or parameters determined in the analytical stations 26 are immediately stored in the input and/or output device 21 or retrievably filed in the database of the laboratory information system 22. Only when all variables or parameters stipulated in the test requests of the order record have been determined, are all the analytical results transmitted in packet form to the user 1 via the data network 41, having been generated in, or converted into, a structured data format which defines both data content and data presentation, for example in an XML format. As the language of interpretation of the 2D barcode, the laboratory data-carrier/data-record description of the German National Association of Panel Doctors may also be used.

For the order record, in particular for the 2D barcode, the PDF417 barcode may be used. This barcode type is also used by the National Association of Panel Doctors as a standard barcode. This will make it easy for read and/or write systems which work with this barcode to decode this code. The PDF417 barcode can be set up with the following parameters:

Resolution: 300 dpi;

Data columns: 12;

Error correction level: 5;

Net dataset in one data block 40: approx. 900, max. 1000 bytes;

Resultant width of one block: approx. 46 mm;

Resultant height (depending on the dataset actually contained): max. approx. 27 mm.

The data blocks 40 in the lower part of the order form 17 can be rotated anticlockwise through 90° and printed at 30 mm intervals starting from the left. The laboratory data-carrier/data-record description of the German National Association of Panel Doctors is defined as follows:

<Field length><Field code><Data><Line separator>

The information on field length is redundant, as a field is separated by a field separator anyway. This means that the field structure may for example be defined as follows:

<Field code><Data><Line separator>

The field code has a four-digit configuration (e.g. 3101=patient's surname).Data (e.g. Huber).Closing separator (Return).

Here it should be expressly pointed out that any differently defined 2D barcode can also be used.

Below are some examples of field codes of the PDF417 barcode and the field descriptions assigned to them:

Code Field Description

8000 Record identifier

8100 Record length

8310 Request ident

8609 Accounting type

3100 Patient's name header

3101 Patient's surname

3102 Patient's first name

3103 Patient's date of birth

3104 Patient's title

3105 Insured-person number

3106 Patient's place of residence

3107 Patient's street

3108 Category of insured Mem/Fam/OAP (MFR)

8405 Patient details

8407 Sex of patient

3201 Surname of original insured (HV)

3202 HV's first name

3203 HV's date of birth

3204 HV's place of residence

3205 HV's street

2002 Sickness fund name

4104 Insurance card number (VKNR)

4106 Cost-unit accounting field (KTAB)

4109 Date of last read-in of insurance card (VK) during quarter

4110 Expiry date

4111 Sickness fund number (IK)

4112 Status of insured according to insurance card (VK)

4113 East/West status according to insurance card (VK)

8403 Tariff

4122 Accounting field

4209 Order/diagnosis/presumption

4217 Panel doctor number of first originator

4218 Referral by panel doctor number

4219 Referral by other doctors

4220 Referral to

4239 Apparent subgroup

4221 Curative/preventive/other remedial treatment/treatment by a Belegarzt [=a GP who also looks after a certain number of patients in a hospital]

4222 Code number O I./O II.

4223 Code number O III.

8610 Private tariff

8601 Name of person to whom invoice should be addressed (RE)

8602 RE's title, first name

8606 RE's place of residence

8607 RE's street

8608 Commentary/file number

8503 Infectious

8510 Pregnancy

8511 How long pregnant

8504 Medication at the time when sample was taken

8512 1st day of last cycle

3622 Patient's height

3623 Patient's weight

8501 Emergency status

8611 Additional diagnosis

8612 Telephone number

8613 Additional recipient

8434 Requests

8410 Test ident

8411 Test designation

8428 Sample material ident

8429 Sample material index

8430 Sample material designation

8431 Sample material specification

8432 Sample collection date

8433 Sample collection time

8520 Quantity of sample material

8521 Unit of measurement

The user 1 is able to access at least one graphic user interface at the input and/or output device 5 at which the analytical results from the laboratory 2 are automatically entered in predetermined data fields. The computer system 4 may be formed by a PC host computer, with the input and/or output device 5 subordinated to the computer system 4. Input of the analysis request and/or suspected diagnosis and/or output of the information catalogue may be effected on at least one Internet page, in particular on an html page presenting the user interface. This html page presenting the user interface cannot be amended by the user 1. The user interface is for example formed by the screen mask, conveniently only for inputting data via the input device 5 and outputting the analytical results in the control and/or processing programme, in which only an interpretation or presentation of the analytical results, especially when predeterminable limit values for analytical results are reached, and a corresponding output for the user 1 is output. To quote an example: treatment or therapeutic measures are output automatically if a lower or upper limit value of e.g. the blood sugar level is reached. These limits assigned to the variables or parameters to be determined and data on treatment or therapeutic measures which have been filed for these limits are retrievably filed in at least one information catalogue.

By virtue of the communication link between the user 1 and the laboratory 2, the user's additional data management device 10 for information catalogues may no longer be necessary. Instead, the user 1 can avail himself via the data network 41 of the data retrievably filed in information catalogues in the database of the laboratory information system 22. These information catalogues can be continually updated by the laboratory 2. The analytical results obtained are likewise retrievably filed in the database in the laboratory information system 22 forming a data archiving facility.

It will also be evident from FIG. 3 that it is possible for a plurality of users 1, say 2 to 100, and a plurality of laboratories 2, say 2 to 10, to be linked for data transmission purposes via the global data network 41, in particular via the Internet, with a central management system 42. This central management system 42 comprises at least one computer system, e.g. a PC, and at least one database, with access e.g. to the database of this central management system 42 restricted to authorized users. Such a body of authorized users may be formed for example by a specialists' association, e.g. a medical association. With such an arrangement, the information catalogues described above, which were retrievably filed at the user's surgery 1 in the data management device 10 and/or at the laboratory 2 in the laboratory information system 22, can be filed solely in the central management system 42.

Such an internet-based solution has the advantage that the lapse of time between removal of the sample and receipt of the analytical result is shortened to the time it takes for the sample to be dispatched and processed in the laboratory 2.

The possibility of accessing the information catalogues retrievably filed in the central management system 42 and/or in the laboratory information system 22, in conjunction with the transmission or availability of data, in particular order records and analytical results, through the data network 41, affords the user 1 the advantage that, firstly: the information catalogues and/or the application software, in particular the user interface, used by the user 1 or by the laboratory 2 can be centrally maintained e.g. by the laboratory 2 and/or the central management system 42. This means that changes, such as changes to the sample containers 13, e.g. blood collection tubes, e.g. a change in the presented reagents, can easily be amended or updated at any time by a central agency for the whole body of users 1 affiliated to the sample management system, e.g. doctors' practices, research centres, laboratories 2, etc. Secondly: it makes it possible for additional analytical options offered by the laboratory 2 to be made available to the user 1 without any problem, so that separate updating is no longer necessary. The possible tests offered by the individual laboratory 2 can therefore be updated and made known to the user 1 on an ongoing basis.

With the aid of a mobile communication device such as the above-mentioned palm-size data terminal or a mobile phone with WAP technology or a radio etc., it is moreover possible for the data to be transmitted to the laboratory 2—again via the data network 41 e.g. the Internet—not only for example by GPs on home visits, but also by emergency doctors and rescue services. In this case, the communication device is equipped in particular with an infra-red interface.

Another possibility besides the above-mentioned coding of data is for patient data in particular to be suitably encoded by the doctor so that the matching of analytical results to the individual patient can ultimately be done only by the doctor himself, so preserving the patient's anonymity. Thus a problem which arises with HIV for example, namely social exclusion of the patient through “leakage” of data, can be virtually eliminated, subject of course to the confidential relationship between doctor and patient being preserved.

Moreover, the computer system 4 makes it possible for hints (including hints for less experienced users) on sample handling during collection to be recorded and displayed, e.g. in the form of a procedure or working instruction. These data forming a procedure or working instruction are filed in the information catalogue as pictorial and/or sound data, and are output via the user interface or control and/or processing programme at the input and/or output device 5.

The information catalogue may be made up of a multitude of data filed in tables and/or registers in the database for each application i.e. each suspected diagnosis and/or analysis request. In an embodiment concerned with the analysis of samples of biological origin, in particular human origin, this information catalogue may contain for example data relating to several specimen container manufacturers and their ranges of different sample containers 13, that is to say for example data on blood sample tubes with different reagents and/or urine sample containers and/or faecal specimen containers and/or tissue specimen containers or the like. The information catalogue may also indicate what kind of sample is required, i.e. what material should be removed for the specific application. In the case of samples of biological origin this material may consist for example of blood and/or serum and/or urine and/or faeces and/or tissue and/or amniotic fluid.

Furthermore, data or instructions may be filed on special conditions for the collection of these samples, such as for example a requirement for the patient to have an empty stomach when the blood sample is taken, or indication of a precise site from which a tissue sample is to be taken, or the exact week of pregnancy for collection of amniotic fluid or the like. Another highly important point is that the information catalogues suggest the variables, in particular the parameters, of a sample that should be determined. For a blood sample, it may be suggested for example that a blood sugar level, cholesterol or triglycerin level or haemoglobin level should be determined. For a faecal specimen, various bacterial cultures might need to be determined, for example. “Variable to be determined” means both the presence of a certain substance and its numerical value, i.e. information for a qualitative and/or quantitative analysis.

Information on manufacturers which may also be included in the information catalogue may comprise for example a reference to a limited shelf-life of sample containers 13 or of the reagents in the sample containers 13, or specific storage conditions, such as a storage temperature for example.

Other data may also be included in the information catalogue, e.g. information regarding sample handling, sample storage, sample processing or sample dispatch.

The information catalogues promulgated may be adapted to special requirements or analytical capabilities of at least one laboratory 2. For example, they may define the sample containers 13 or sample container manufacturers to be used by the user 1. For example, the analytical options offered may be those which are actually carried out by this particular laboratory 2. Another possibility is that, depending on the extent of the analyses to be performed, the database (of the user 1 or of the central management system 42) chooses the available laboratory 2 which is best able to cope with the analytical tasks requested by the user 1, and suggests that laboratory through the medium of the information catalogue. It is also possible for the information catalogue to suggest that the samples be sent to different laboratories 2 for further analysis so that the samples can be processed more quickly.

In order to facilitate the generation of the information catalogue in the database in the data management device 10, and/or to prevent the transmission of unnecessary data, it is possible for the sample containers 13 available at the premises of the user 1 to be ascertained by means of the computer system 4, or made known to the computer system 4, and for this information to be forwarded to the database, for example upon transmission of a suspected diagnosis or analysis request. An information catalogue adapted to the available sample containers 13 is then issued by the database. The information on available sample container manufacturers may for example be orientated to the sample container 13 used by the user 1, with or without limitation of the sample container manufacturer's product range.

At this point, it should be expressly pointed out that the information catalogue transmitted by the database merely constitutes a lead or suggestion as to the materials to be collected, variables to be determined and/or sample containers 13 to be used for a given application. This information catalogue can be freely amended by the user 1 to suit his preferences or requirements, that is to say, that the materials actually collected and to be analysed and/or sample variables to be determined can be changed, and in particular augmented or reduced, to suit requirements. The information catalogue provides the user 1 with a more convenient and above all a reliable means of generating order data for samples to be analysed, and expediently contains all data and directions which the user 1 needs to be aware of.

The information catalogue is generated from a multitude of possible information catalogues, or compiled from a multitude of data stored in tables and/or registers in a database which is stored locally in the computer system 4 of the user 1 or is called up via the data network 41 by a server computer of the central management system 42. If the database is filed on a server or PC that can be reached via the data network 41, it is possible for it to be serviced, and in particular for information catalogues (or the tables and/or registers for generating the information catalogues) to be updated, by at least one central management system 42. It is also possible to let the database be updated by several central management systems 42, in which case it is expedient to have the range of possible sample containers 13 updated by the sample container manufacturer(s), and the sample variables for analysis updated by a specialist association and/or by the laboratory 2.

The information catalogue may also contain pictorial and/or sound data whose output can be controlled via the user interface.

When a relatively large amount of sample material is to be collected, it is also possible for the information catalogue to suggest that several similar sample containers 13 be used to contain this large quantity. This is also possible when several variables of the sample or material are to be determined, and can considerably shorten analysis times in the laboratory 2 and/or leave over a sufficient amount of sample material in the laboratory 2 for any subsequent analyses that may be required. As the computer system 4, the user 1 may use a standard commercial PC possessing a data network connection, in particular an Internet connection, that is e.g. an html browser which uses a protocol, in particular http. The input of the clinical picture or suspected diagnosis and/or output of the information catalogue may be effected via an Internet page, in particular an html page.

The input of the suspected diagnosis on the internet page can be set up so that it is subject to a system of controls. Then, depending on the data entered and fields filled in, further selection fields or input fields can be displayed or enabled, or as the case may be, barred or visually blocked out. For instance, for samples from male patients the “pregnant yes no” selection field can be blocked out or deactivated.

The further data of the information catalogue may also be supplemented for example by empirical values. For example, the time usually required for carrying out the analyses may be stated. This is particularly important in the case of bacteriological investigations, as such analyses often involve waiting times of several weeks, of which the user 1 needs to be made aware.

The problem which lies at the basis of the independent inventive solutions can be inferred from the description.

Above all, the individual embodiments shown in FIGS. 1; 2; 3 can form the subject-matter of independent solutions according to the invention. The relevant problems and solutions according to the invention can be inferred from the detailed description of these figures.

### List of Reference Numbers

1 user

2 laboratory

3 patient

4 computer system

5 input and/or output device

6 terminal

7

8 data line

9 data management device

10 data management device

11 acquisition device

12 printer device

13 sample container

14 data carrier (sample container)

15 barcode

16 data carrier

17 order form

18 container

19 mounting device

20 order recording point

21 input and/or output device

22 laboratory information system

23 system control centre

24 data line

25 sorting unit

26 analytical station

27 acquisition device

28 archiving facility

29 portion

30 portion

31 data field

32 data field

33 data field

34 data field

35 data field

36 data field

37a data field

37b data field

37c data field

38 data field

39 data field

40 data block

41 data network

42 central management system

1. Method for taking a sample from a system, wherein at least one analysis request for the sample has been formulated before the start of the method and is input by a user into a computer system of the user and a connection is then established between the computer system and a database, wherein an information catalogue is compiled from data in the database on the basis of the analysis request and is suggested, or at least partially amendably presented, to the user via an input and/or output device of the computer system and the type of the at least one sample container to be used is suggested on the basis of the information catalogue.

2. Method for the generation of at least one order record for at least one sample to be analysed, wherein data on a system, for example a patient, are recorded in a computer system and a unique identification number for the system, for example the patient, is determined by the computer system and at least one analysis request for the sample is entered into a computer system of the user and a connection is made between the computer system and a database, characterized in that the database suggests an information catalogue in particular according to claim 1 which is germane to the entered analysis request and in which the data filed for the entered analysis request and output at an input and/or output device in the user domain are if necessary supplemented or amended by the user and the test requests for a laboratory analysing the at least one sample are generated from at least some of these data and in that at least one common, standard, machine-processable order record is generated by the computer system from the test requests and output data regarding the at least one sample container stipulated for removal of the sample and the identification number allocated to the system by the computer system and an order number stipulated by the user, and is transmitted to the appointed laboratory.

3. Method according to claim 1, wherein at least one suspected diagnosis which has been made before the start of the method on the basis of at least one condition of the system is input by a user into a computer system of the user and a connection is then made between the computer system and a database; an information catalogue then being determined from data in the database on the basis of the suspected diagnosis and this information catalogue being suggested, or at least partly amendably presented, to the user via an input and/or output device of the computer system and the at least one type of sample to be taken being suggested on the basis of the information catalogue.

4. Method according to claim 1, wherein the input of the analysis request and/or suspected diagnosis and output of the information catalogue are effected at the input and/or output device by means of at least one user interface, e.g. a screen mask.

5. Method according to claim 1, wherein data are filed in the information catalogue on the at least one sample container to be used by the user, in particular data relating to a manufacturer and/or to the sample container of at least one manufacturer's range of sample containers that is to be used.

6. Method according to claim 3, wherein the type of sample to be taken suggested by the information catalogue is a blood and/or serum and/or urine and/or faecal and/or tissue and/or amniotic fluid sample.

7. Method according to claim 1, wherein data are filed in the information catalogue on special sampling conditions such as a specific sampling temperature, or the need for the patient's stomach to be empty when a blood sample is taken.

8. Method according to claim 1, wherein at least one sample variable to be determined, in particular a parameter, for example a blood sugar level, is suggested by the information catalogue.

9. Method according to claim 1, wherein data regarding sample handling, sample storage, sample processing, sample dispatch or an anticipated waiting time to receipt of analytical results are filed in the information catalogue.

10. Method according to claim 1, wherein data regarding special requirements or analytical facilities of at least one laboratory and naming at least one particular sample container manufacturer to be used or divulging the variables that can be determined by the laboratory are filed in the information catalogue.

11. Method according to claim 1, wherein a possible choice of laboratory based on the extent of the variables to be determined is divulged in the information catalogue.

12. Method according to claim 1, wherein the type and/or number of sample containers delivered to the user are identified or electronically recorded via the acquisition device and these read-out data regarding the type and/or number are retrievably filed in the user's database and in that the type and/or number of sample containers from which the user can choose are output at the input and/or output device of the computer system.

13. Method according to claim 12, wherein information on the sample containers from which the user can choose is forwarded to the database upon transmission of the analysis request and/or suspected diagnosis.

14. Method according to claim 1, wherein the data suggested in the information catalogue are supplemented, amended or accepted by the user and test requests for the laboratory are generated therefrom.

15. Method according to claim 14, wherein the test requests are sent to the appointed laboratory as advance information, independently of the sample container.

16. Method according to claim 1, wherein the test requests are recorded on a data carrier.

17. Method according to claim 16, wherein the data carrier contains a graphic data record, in particular in the form of a 2D barcode.

18. Method according to claim 17, wherein the graphic data record is placed on an order form.

19. Method according to claim 17, wherein the data record image is split up into a number of separate data blocks of specified memory size.

20. Method according to claim 19, wherein the memory size of one data block is between 100 and 1300 bytes, in particular between 600 and 1100 bytes, e.g. 950 bytes.

21. Method according to claim 14, wherein the test requests are recorded in a transponder and/or magnetic stripe.

22. Method according to claim 1, wherein a PC is used as computer system, the database is filed on a server connectable to a data network, in particular the Internet, and the input of the analysis request and/or suspected diagnosis and/or the output of the information catalogue are performed on at least one Internet page, in particular on an html page constituting the user interface.

23. Method according to claim 1, wherein pictorial and/or sound data are filed in the information catalogue and their output is controlled via the user interface.

24. Method according to claim 1, wherein the database is maintained via the, preferably global, data network, in particular the Internet, by at least one central management system, with, in particular, data concerning the range of possible sample containers updated by the sample container manufacturer(s), and/or data concerning the sample variables for analysis updated by a specialist association or by the laboratory.

25. Method according to claim 1, wherein the number of similar sample containers to be used and/or the quantity to be collected are suggested by the information catalogue.

26. Method according to claim 1, wherein treatment or therapeutic measures for the entered suspected diagnosis are suggested by the information catalogue.

27. Method according to claim 1, wherein the suggested sample container is distinctively identified through the container data before it is actually used e.g. filled, the container data are read into the computer system via an acquisition device when the sample container is used, and after the container data identifying the sample container are read in, a programme, preferably menu-driven, is opened, in particular a control and/or processing programme or a user interface.

28. Method according to claim 1, wherein in the menu-driven programme, in particular a control and/or processing programme, the test requests for the at least one sample for analysis are recorded, the container data of the stipulated sample container (which have already been read in) are raised and the data on the system, for example the patient data, are read out of the database or recorded by the user and (if required) the order number for this order is issued by the computer system and/or at least one common, standard, machine-processable order record is thereupon generated from the test requests, container data, data on the system and (if required) the order number.

29. Method according to claim 1, wherein the order record is supplemented by the inclusion of data identifying the user and/or data identifying the person who takes the sample.

30. Method according to claim 1, wherein the order record which has been generated is stored on a physical data carrier, in particular a magnetic stripe and/or chip and/or transponder and/or rewritable plastic card and/or floppy disk and/or CD-ROM and/or order form.

31. Method according to claim 30, wherein the data carrier is placed in and/or on the sample container, or in and/or on a mounting device holding at least one sample container.

32. Method according to claim 30, wherein the data carrier carries the order record in graphic form, in particular in the form of a 2D barcode.

33. Method according to claim 30, wherein the order record image is split up into a number of separate data blocks of specified memory size.

34. Method according to claim 33, wherein the memory size of one data block is between 100 and 1300 bytes, in particular between 600 and 1100 bytes, e.g. 950 bytes.

35. Method according to claim 1, wherein the order record is recorded in the material of the sample container and/or mounting device and/or physical data carrier for holding the at least one sample container.

36. Method according to claim 32, wherein the laboratory data-carrier/data-record description of the German National Association of Panel Doctors is used as the language of interpretation of the 2D barcode.

37. Method according to claim 1, wherein the order record which has been generated is transmitted into a data network, in particular the Internet, having been generated in, or converted into, a structured data format which defines both data content and data presentation, for example in an XML format.

38. Method according to claim 1, wherein a user interface is amended in successive method-steps, e.g. transfer of patient data; input of the analysis request and/or suspected diagnosis; output of the information catalogue; additions and/or amendments and/or acceptance of data of the information catalogue for the issue of test requests; generation of the order record, in particular a coherent mask-sequence is run through.

39. Method according to claim 1, wherein method-steps are initiated via at least one input and/or output device of the computer system, with menu-driven user-guidance.

40. Method according to claim 1, wherein an order form for the dispatch of the sample container or mounting device is generated from the order record, the design, in particular, of the order form being governed by the order record, for example by the patient's sickness insurance scheme.

41. Method according to claim 1, wherein the patient data are (as is known in itself) read from a machine-readable card carried by the patient, e.g. an insurance card, with a magnetic stripe and/or chip and are transferred into the computer system, and a distinctive code recorded on a carrier and staying with the patient is allocated to these patient data for the subsequent identification of the patient, especially a patient who is being treated in hospital.

42. Method according to claim 41, wherein the code recorded on the carrier, e.g. a bracelet, is presented in the form of graphic data, in particular a 1D or 2D barcode.

43. Method according to claim 1, wherein an order number, in particular a serial order number, is issued, and forms part of the order record.

44. Method according to claim 43, wherein part of the container data is used as order number.

45. Method according to claim 1, wherein an incoming check is made in the laboratory by comparing the order record with electronically stored or transmitted data.

46. Method according to claim 1, wherein automatic sorting and/or allocation to analytical stations in the laboratory is effected on the basis of the data in the order record.

47. Method according to claim 1, wherein on completion of the analysis the variables determined, e.g. the analytical results, are made available to the user via a data network, in particular via the Internet.

48. Method according to claim 1, wherein the identification data on the user and/or on the person who takes the sample include data relating to the doctor's practice or hospital or department to which the person belongs.

49. Method according to claim 48, wherein the identification data together with biometric features, in particular a fingerprint and/or an iris form and/or a facial shape, are taken and stored on a once-only basis and are used for the subsequent recognition of the person, whereupon the identification data are read out and made available for the generation of the order record.

50. Use of the method according to claim 1, for the taking of samples of biological origin, in particular human origin.

51. Use of the method according to claim 1, for the taking of samples for the determination of environmental pollution or ecological damage.

52. Use of the method according to claim 1, for the taking of samples for the determination of liquid composition, in particular water quality.
